(19) United States
(12) Patent Application Publication
(10) Pub. No.: US 2009/0042240 A1
(43) Pub. Date: Feb. 12, 2009

(54) RAPID DETECTION OF ANTIMICROBIAL DRUG RESIDUES

(76) Inventors: Jacobus Stark, Rotterdam (NL); Angelique de. Rijk, Ridderkerk (NL)

Correspondence Address: NIXON & VANDERHYE, PC, 901 NORTH GLEBE ROAD, 11TH FLOOR, ARLINGTON, VA, 22203, US

(21) Appl. No.: 12/280,256

(22) PCT Filed: Feb. 21, 2007
(86) PCT No.: PCT/EP2007/051651

§ 371 Date: Aug. 21, 2008

(30) Foreign Application Priority Data
Feb. 24, 2006 (EP) 06075431.4

(51) Int. Cl. C12Q 1/18

(52) U.S. Cl. 435/32

(57) ABSTRACT

The present invention relates to a novel method for the rapid detection of the presence or absence of antimicrobial drug residues belonging to the class of the quinolones in liquid samples such as milk, meat juice, serum, urine, blood, eggs or extracts obtained from animal tissues or food products.

### FIELD OF THE INVENTION

The present invention relates to a novel method for the rapid detection of the presence or absence of antimicrobial drug residues belonging to the biochemical class of the quinolones in liquid samples such as milk, meat juice, serum, urine, blood, eggs or extracts obtained from animal tissues or food products.

### BACKGROUND OF THE INVENTION

Antimicrobial drugs are widely used to treat diseases in animals; the use of veterinary pharmaceuticals in modern farming is common practice. Antimicrobial drugs are not only applied as medication, but are also widely used to prevent illness, enhance the growth of animals or improve the feed efficiency. The antimicrobials are administrated by injection or may be added to the feed or drinking water.

Intensive animal breeding for food production has increased the use of antimicrobial agents. The presence of antimicrobial drugs in food is a growing concern among consumers, allergic reactions and negative effects on the intestinal human flora are well known. Another risk is the development of resistant bacteria; this risk consists of the possible transfer from animals to humans of resistant zoonotic bacteria such as Salmonella, Campylobacter or Escherichia coli species. In the particular case of quinolones there are additional concerns about the development of bacterial resistance, since quinolones are also important in the treatment of human infections.

The quinolone class of antibacterial agents comprises a large group of synthetic heterocyclic aromatic compounds. Quinolones inhibit DNA synthesis by interacting with two key enzymes needed for DNA replication: DNA gyrase and topoisomerase IV. The quinolone class drugs are specifically bacteriostatic and bactericidal for gram-negative bacteria. The development of the first generation of quinolones (e.g. nalidixic acid, cinoxacin and oxolinic acid) started in 1965; later the more effective fluoroquinolones (e.g. enrofloxacin, ciprofloxacin, danofloxacin, marbofloxacin and sarafloxacin) were developed. In two decades this group of synthetic antimicrobials moved from a relatively unimportant group of drugs to a class with worldwide applications in animal feed, animal drinking water and in animal/human medication.

Most veterinary drugs enter the human food chain through animal products such as meat, milk and eggs. Maximum Residue Limits (MRL's) for the widely used members of the quinolones class have been set by authorities such as the European Community; quinolones that are not allowed may never be present in animal products (zero tolerance). For most drugs a withdrawal period is determined, during this period drug residues decrease to a level below the MRL. Due to individual differences between animals or because of disturbance of the animal metabolism, even after the withdrawal period too high concentrations of the quinolone may be present in the animal tissues. Also withdrawal procedures may not be applied correctly.

Tests programs to determine the presence of quinolone residues in slaughter animals (e.g. cattle, pigs, and poultry), meat, liver, kidney, eggs, honey, fish, shrimps, milk or animal derived food products are mostly executed by governmental control laboratories. In addition also slaughterhouses, meat processing industry, dairy companies, retailers and supermarkets may examine animals or animal derived food products on the presence of unwanted compounds. To apply these studies in a proper way, rapid reliable and easy-to-use tests are required.

The current diagnostic methods to detect quinolones in food matrixes are extremely expensive, time consuming and require high professional skills; methods of analysis include High Performance Liquid Chromatography (HPLC) and mass spectrophotometry. Individual compounds can be detected using commercial tests based on the well known antibody technology. However such ELISA tests can only detect one or a very limited number of compounds and are not suitable as screening assay for the detection of quinolones. Besides this, these tests are too expensive for general use as screening test.

For screening purposes usually microbial inhibition assays are used. These are carried out by placing pieces of animal tissue (e.g. pieces of muscle tissue or kidney) or paper disks saturated with e.g. milk, meat fluid, kidney fluid, urine or blood on agar cultures of a sensitive bacterium species, e.g. Bacillus subtilis, Micrococcus luteus or Escherichia coli. The plates are incubated for a sufficient period at the optimal growth temperature of the bacterium. After incubation of mostly at least 18 hours, the presence and size of an inhibition zone indicates the presence of antimicrobial compounds in the sample.

The European Four-Plate test uses media seeded with Bacillus subtilis or Micrococcus luteus. Okerman and Van Hoof (Evaluation of the European Four-Plate test as a tool for screening antibiotic residues in meat samples from retail outlets, Journal of AOAC International, V81, No. 1 (1998), page 51-56) concluded that this official method was not sensitive enough to detect quinolones.

Choi et al. described a method for detecting quinolones using Escherichia coli strains (Determination of fluoroquinolone residues in animal tissues using Escherichia coli as indicator organism, Journal of AOAC International, V82, No. 6 (1999), page 1407-1412). However the sensitivities found were insufficient to meet the MRL's stated by the European Community. The detection limits were: enrofloxacin (50 ppb, MRL=100 ppb); ciprofloxacin (30 ppb, MRL=100 ppb); sarafloxacin (250 ppb, MRL=100 ppb); difloxacin (250 ppb, MRL=10 ppb).

On a similar way an agar diffusion method was developed by Chin-En Tsai and Kondo (Improved agar diffusion method for detecting residual antimicrobial agents, Journal of Food Protection, V64 (2001), No. 3, page 361-366). In this study a sensitivity of 3120 ppb was reported for nalidixic acid and 200 ppb for oxolinic acid.

Suhren et al. reported on the detection of quinolones in milk (Detection of residues of quinolones in milk, Residues of veterinary drugs in food—Proceedings of the Euroresidue III Conference, Veldhoven (NL) 6-8—May 1996, pages 917-921). In this study, sensitivities of 2 ppb, respectively 5 ppb were reported for enrofloxacin and ciprofloxacin when samples of Escherichia coli ATCC 11303 frozen in 10% glycerol were used as a testing organism. In an additional study the same authors reported the following sensitivities: ciprofloxacin: 3 ppb; enrofloxacin: 6 ppb; danofloxacin: 8 ppb; sarafloxacin: 15 ppb. (G. Suhren 1997, Microbiologischer Hemmstofftest mit Escherichia coli zum nachweis von chinolon-rückstanden in Milch, Arbeitst. des Deutsches veterinairmedizinische Gesellschaft Lebensmittelhygiene, Proceedings Conference Garmisch Partenkirchen, 1997). In this study the microorganism was frozen in skimmed milk and cells were washed with saline prior to freezing.

An object of the present invention can therefore be seen as the provision of an alternative test system that provides equal or better results as the tests mentioned above. Preferably the test should have at least one of the below advantages. Ideally, a commercial screening test to detect quinolones should meet the following requirements:
1. Rapid
2. A high sensitivity for a wide range of quinolones
3. Easy-to-use, even by unskilled people
4. Reliable
5. Cheap
6. A shelf life of at least one month.

The current diagnostic methods to detect quinolones do not fulfill these requirements. Diagnostic methods such as HPLC dot not fulfill points 1, 2, 3 and 5; screening tests in which inhibition zones are determined do not meet any of the requirements; the tests described by Suhren are too slow (19-21 hours), have a shelf life of maximum 14 days, while it is unclear if a wide range of quinolones can be detected.

It may be concluded that the prior art methods leave to be desired with respect to at least one of the above requirements. As a result of this, food products and slaughter animals are hardly examined on the presence of quinolones. Further from an economical and logistic point of view it is in today's industry unacceptable to wait more than one working day before the test results are known and e.g. a slaughtered animal can be processed; in practice producers often do not wait for the results, the animals or food products are already processed before the results of the test are known. All this may lead to unacceptable amounts of quinolones in the human food chain.

### DETAILED DESCRIPTION OF THE INVENTION

The test methods described in this invention fulfill all six requirements described above, now liquid samples can easily be examined on the presence of quinolones compounds. Surprisingly, we found a method to detect within 10 hours, or even within 5-7 hours, all important compounds belonging to the group of quinolones known today around or below the present MRL's.

In a first aspect of the invention there is thus provided a method for the rapid determination of the presence of residues belonging to the group of quinolones in for example milk, meat juice, blood, serum, urine, eggs, animal tissues and food products which comprises the steps of
(1) Taking a liquid sample such as milk, blood or urine; optionally the liquid sample can be purified using a solvent extraction. A liquid sample from e.g. animal tissue or a food product can be obtained by using well-known methods such as squeezing or solvent extraction;
(2) Adding said liquid sample to a test medium containing a sensitive test organism selected from strains of gram-negative bacteria;
(3) Incubating the test together with the contacted sample at a suitable growth temperature of the microorganism;
(4) Detecting the growth of said microorganisms after less than 10 hours, preferably after 5-7 hours, of incubation by measuring e.g. the color change of an indicator present in the test medium.

According to one embodiment the sensitive test microorganism belongs to the group of gram-negative bacteria, preferably a strain of Escherichia coli, more preferably Escherichia coli ATCC11303. For the purpose of the invention vegetative cells or spores may be used from all bacterial species which are sufficiently sensitive to the quinolones to be detected.

Surprisingly we found that when instead of frozen microorganisms (as described by Suhren), cells or spores in dried form are applied, the incubation time of the test was reduced considerably. So this invention includes microbial tests for the detection of quinolones in which dried cells or spores, preferably freeze-dried cells or spores, are applied; alternatively the test may contain freshly prepared cells. The detection levels of the broad-spectrum tests included in this invention are as described in Table 1.

For use in accordance with the invention the test contains 10³-10¹⁰ colony forming units (CFU)/ml of medium, more preferably 10⁴-10⁸ CFU/ml of medium and most preferably 10⁵-10⁷ CFU/ml of medium. The bacterial cultures may be prepared according to known methods.

The growth medium is preferably a medium in which the microorganism of the test can easily grow; the medium contains nutrients to enable the multiplication of the test organism in the absence of antimicrobial compounds. Suitable nutrients are for example assimilable carbon sources (monosaccharide/disaccharides/oligosaccharides such as e.g. dextrose, glucose, lactose, maltose, sucrose or trehalose), assimilable nitrogen sources (e.g. peptone or amino acids) and sources of growth factors, vitamins and minerals (e.g. yeast extract). An example of a suitable medium for Escherichia coli strains is Plate Count Broth. The medium also may contain substances to influence the sensitivity of the test organism in a positive or negative way.

In a preferred embodiment the test medium comprises a disaccharide. Preferably said disaccharide is maltose, sucrose (saccharose) or trehalose.

We also surprisingly observed that a test medium with a pH of 6.0 to 8.0, preferably a pH of 6.5 to 7.5 may advantageously be used in order to reduce the incubation period. The test medium may be optionally buffered.

According to another embodiment the media applied in the test methods described in this invention are not solidified by e.g. agar. Up to now it was supposed that a pre-incubation of 1 hour and the removal of the sample before incubation as described by Suhren were required. We surprisingly found that tests meeting all requirements can be obtained using a liquid medium, which means there is no need to use a solidified medium, e.g. by using agar. Therefore the test medium described in this invention is a liquid to which the test organisms and the sample are added before incubation. By doing so, the incubation time of the test may be reduced.

In a second aspect, this invention includes a composition containing dried bacterial cultures, dry medium compounds such as nutrients, color indicators, buffer compounds and substances to influence the sensitivity of the test. The dry composition can be e.g. a powder, granulate or tablet which can be added directly to the liquid sample or alternatively may be used to prepare a liquid test medium by dissolving the tablet first in e.g. water after which the liquid sample is added to the liquid test or visa versa. The dried bacterial cultures are preferably lyophilized cells and may be formulated with the medium compounds or added separately before incubation. For the purpose of the present invention, a dry composition is understood to be a composition wherein the amount of water present is less than 4%, preferably less than 2%, more preferably less than 1%, most preferably less than 0.5%, of the total weight of said composition. Suitable color indicators are pH- and/or redox indicators. Examples of suitable indicators are acid blue 120, acid orange 51, acid yellow 38, alizarin acid, alizarin blue, azure A, azure B, basic blue 3, brilliant black, brilliant cresyl blue, brilliant crocein MOO, brilliant yellow, bromocresol green, bromocresol purple, bromophenol blue, bromophenol red, bromothymol blue, chlorocresol green, Congo red, m-cresol purple, gallocyanine, indigo carmine, Janus green B, litmus, methylene blue, neutral red, Nile blue, Nile blue A, nitrazol yellow, o-nitrophenol, p-nitrophenol, 1,10-phenanthroline, phenolphthalein, phenol red, safranine O, thionin, thymol blue, toluidine blue and xylenol blue.

Optionally all compounds such as the test organism, the nutrients, the buffer and the indicator components may be added as a separate source, for example by application of a tablet, paper disc, granulate or dry powder.

In a third aspect the invention provides a test unit for carrying out the method of the invention comprising a container in which the dried or freshly prepared cells or spores together with the test medium are mixed with the sample. Examples of containers are tubes (single, multiple or blocks), microtiter plates, sample bottles and plates. Alternatively the dry composition may also be attached to strips, dipsticks, paper disks, and the like.

The test has to be stored in such a way that the test organism does not multiply before incubation of the test. This may be achieved by depriving the organism of nutrients until the test is carried out and/or by maintaining the test units at a sufficiently low temperature and/or by adding the nutrients/test organism separately and/or by drying the test or test organisms.

The incubation temperature is dependent on the circumstances, e.g. the microorganism used and the nutrients and temperature applied. When using dried, preferably freeze-dried, or fresh Escherichia coli cells, suitable incubation temperatures are about 30° C.-40° C., more preferably 30° C.-38° C. and most preferably 36° C.-38° C. Incubation periods within which reliable results are obtained are relatively short and vary from 1-10 hours, preferably from 3-9 hours, more preferably from 4-8 hours, most preferably from 5-7 hours. The incubation can be executed by placing e.g. the test tubes in an incubator or water-bath set at or near the optimal temperature.

The growth of the test organism can be detected by using well known methods, preferably by detection of acid production, oxygen consuming or other metabolic activities of the test organism, which can be detected by e.g. a color change of the test in the presence of a color indicator. Preferably one or more redox or acid-base color indicators are used. Examples of suitable indicators are bromocresol purple, phenol red, brilliant black, methylene blue, toluidine blue, neutral red, Nile blue, acid blue 120, acid orange 51, acid yellow 38, alizarin acid, alizarin blue, azure A, azure B, basic blue 3, brilliant cresyl blue, brilliant crocein MOO, brilliant yellow, bromocresol green, bromophenol blue, bromophenol red, bromothymol blue, chlorocresol green, Congo red, m-cresol purple, gallocyanine, indigo carmine, Janus green B, litmus, Nile blue A, nitrazol yellow, o-nitrophenol, p-nitrophenol, 1,10-phenanthroline, phenolphthalein, safranine O, thionin, thymol blue and xylenol blue.

In the region where the concentration of quinolones is below the limit of detection of the test, inhibition of the growth will not occur so that the medium is influenced by the growth of the microorganism, and the indicator changes its color. When bromocresol purple is applied, the test medium will turn from purple into yellow. The region where the concentration of the quinolones is above this level, inhibition of growth occurs and the indicator will not change color. The color can be determined visually or by using equipment such a scanner system and suitable software as described in EP 953149.

The test method, composition and unit have been developed especially to examine animal derived products on the presence or absence of quinolones. In practice slaughter or consumption animals such as poultry, cattle, pigs, sheep, fish and shrimps are examined by determining quinolone residues in muscle tissue, kidneys, organs, blood, blood serum or urine. Samples can also be obtained from consumption meat, processed meat products or food products containing animal derived ingredients such as baby food and ready-to-eat-meals. Products produced by animals such as milk, eggs and honey can also be examined.

Liquid samples such as milk, urine, blood or drip liquid of meat may be added to the test medium directly; from solid samples such as muscle tissue, a liquid sample can be obtained using well-known methods such as heating and/or squeezing using e.g. a garlic press. The samples obtained can optionally be purified using e.g. a centrifugation step or a filter. Alternatively a sample can be obtained or purified using a solvent extraction method. An example of a suitable solvent is acetonitrile/acetone (70:30, v/v), but of course any suitable solvent and any suitable extraction method known in the art may be used.

### EXAMPLES

### Example 1

### Extraction Procedure

An example of an extraction procedure to obtain a liquid sample from muscle tissue is:
1. Weigh out 4 grams of muscle tissue;
2. Add 10 ml acetonitrile/acetone 70:30 and 5 grams of anhydrous sodium sulfate powder and homogenize for 30-40 seconds using an Ultra Turrax;
3. Place in an ultrasonic bath for 5-10 minutes, then vortex mix for 30-40 seconds;
4. Centrifuge for 10-12 minutes at 4200 G at 4° C.;
5. Decant supernatant into a tube and reduce the volume under nitrogen at 35-40° C. to ˜200 μl;
6. Add 50 μL of 0.5 M NaOH;
7. Adjust to 600 μl with Lab Lemco Broth and place in an ultrasonic bath for 5-10 minutes, then vortex for 1-2 minutes;
8. Apply 100 μl of liquid sample to the test ampoule

### Example 2

### Detection of Quinolones Using a Liquid Based Test

In this experiment a liquid based microbial screenings test for the detection of quinolones is described. The quinolone medium used in this experiment is prepared by dissolving the following compounds in 1 liter of demineralized water:
8.5 gram of Plate Count Broth (PCB) (Difco, cat. no: 275120);
20 gram of NaCl (Merck, cat. no: 106404);
0.042 gram of Bromocresol purple (Merck, cat. no: 103025).

Escherichia coli strain ATCC 11303 was grown using well-known methods. The test medium was prepared by adding Escherichia coli cells to the quinolone medium to a final concentration of approximately 10⁵ cells/ml. The test was prepared by addition of 250 μl of medium containing the test organism in each test ampoule. Spiked samples were prepared by adding different concentrations of the quinolones described in Table 1 to demineralized water. 100 μl of the spiked sample was added to the test ampoule, a negative control (water without quinolones) was included. The tests were incubated at 37° C. for 4 hours and 45 minutes. The tests were read at the time the test incubated with the negative control sample (water without quinolones) changed color from purple to yellow. The results are presented in Table 1.

TABLE 1
Detection limits for quinolones

| Analyte | Detection level | Lowest official MRL* |
|---|---|---|
| Enrofloxacin | 5 | 100 |
| Ciprofloxacin | 5 | 100 |
| Danofloxacin | 5 | 30 |
| Oxolinic acid | 50 | 100 |
| Flumequin | 100 | 400 |
| Sarafloxacin | 10 | 10 |
| Enoxacin | 50 | —** |
| Nalidixic acid | 1000 | —** |
| Norfloxacin | 25 | —** |
| Ofloxacin | 5 | —** |
| Cinoxacin | 2000 | —** |

**no MRL available yet

These results demonstrate that with the test and method described in this example all important quinolones can be detected within 5 hours at or below the official MRL's. In addition also many other important quinolones can be detected at low concentrations.

### Example 3

### Detection of Quinolones Using a Dry Test (Dried in Batches)

In this experiment a dry microbial screening test for the detection of quinolones is described. The dry test contained the following compounds (after dissolution in one liter of liquid):
8.5 gram of Plate Count Broth (PCB) (Difco, cat. no: 275120);
20 gram of NaCl (Merck, cat. no: 106404);
0.042 gram of Bromocresol purple (Merck, cat. no: 103025).

The test was prepared by dissolving PCB (17 g), NaCl (40 g) and Bromocresol purple (0.084 g) in water (1 L). The medium thus obtained was sterilized for 15 min at 121° C., and a solution of 20% maltose (Merck, cat. no: 105911) was added 1:1 to the medium to obtain a final concentration of 10% maltose. To this were added Escherichia coli cells (10⁶-10⁷ cells/ml) and the mixture was lyophilized. The test was prepared by dissolving the lyophilized components in demineralized water followed by mixing for 15 minutes. Quantities of 250 μl of this solution were added to the test ampoules. Spiked samples were prepared by adding different concentrations of the quinolones described in Table 2 to demineralized water. 100 μl of the spiked sample was added to the test ampoule, a negative control (water without quinolones) was included. The tests were incubated for approximately 5 hours. The results were read at the time the test incubated with a negative control sample (water without quinolones) changed color from purple to yellow. The results were similar to the results obtained in Example 2.

These results demonstrate that with the test and method described in this example all important quinolones can be detected within 5 hours at or below the official MRL's. In addition also many other important quinolones can be detected at low concentrations.

### Example 4

### Detection of Quinolones Using a Dry Test (Dried in Ampoules)

This experiment was conducted as described in Example 3, but in this experiment the mixture was lyophilized in glass ampoules in quantities of 500 μl. The lyophilized components are reconstituted by addition 500 μl demineralized water to the test ampoule and mixing for 1 minute. The test is conducted according to Example 3 with as deviation the addition of 200 μl (spiked) sample instead of 100 μl. Results are similar to the results obtained in Example 2.

### Example 5

### Detection of Quinolones Using a Dry Test (Sucrose Instead of Maltose)

This experiment was conducted as described in Examples 3 and 4, but with sucrose (Merck, cat. no: 1.07651.1000) instead of maltose. Concentration and addition of sucrose was similar to maltose. With this method, detection limits comparable to the results in Example 2 were obtained after 8 hours and 30 minutes of incubation.

### Example 6

### Detection of Quinolones Using a Dry Test (Trehalose Instead of Maltose)

This experiment was conducted as described in Examples 3 and 4, but with trehalose (CalBiochem, cat. no: 625625) instead of maltose. Concentration and addition of trehalose was similar to maltose. With this method results are similar to the results obtained in Example 2.

### Example 7

### Detection of Quinolones in Chicken Muscle by Using a Solvent Extraction Method

In this experiment a mixture of acetonitrile/acetone is used to extract antimicrobial compounds out of the sample, further the experiment is executed as described in Example 2. The sample is obtained as follows:
1. Weigh out 4 g (±0.03) of sample into a 50 ml plastic disposable centrifuge tube;
2. Add 10 ml acetonitrile/acetone 70:30 (v/v);
3. Add 5 g of anhydrous sodium sulfate powder;
4. Homogenize for 30-40 seconds using an Ultra Turrax;
5. Place in an ultrasonic bath for 5-10 minutes, then vortex mix for 30-40 seconds;
6. Centrifuge for 10-12 minutes at 4200 G at 4° C.;
7. Decant supernatant into a disposable glass test tube and reduce the volume under nitrogen at 35-40° C. to 200 μl;
8. Add 50 μl of 0.5 M NaOH;
9. Adjust to 600 μl with Lab Lemco Broth (can be performed using a 1 ml plastic syringe and needle);
10. Place in an ultrasonic bath for 5-10 minutes, then vortex for 1-2 minutes;
11. Check the pH of each sample at this point (pH should be between 5 and 7);
12. Apply 100 μl of extract to the test ampoule;
13. Incubate the test as described in Example 2.

The results obtained were comparable with the results obtained in Example 2. However with this extraction method oxolinic acid and flumequin could be detected at the lower concentration of 10 μg/kg.

1. Method for determining the presence of residues belonging to the group of quinolones in a sample comprising the steps of:
(a) Contacting the sample with a test organism selected from the group consisting of gram-negative bacteria in order to obtain a test mixture;
(b) Incubating the test mixture at a suitable growth temperature of the test organism;
(c) Detecting the growth of the test organism after less than 10 hours.

2. Method according to claim 1 wherein the test organism is Escherichia coli.

3. Method according to claim 2 wherein the test organism is Escherichia coli ATCC1 1303.

4. Method according to claim 1 wherein the test organism is added to the sample in a dried or freshly prepared form.

5. Method according to claim 1 wherein the test mixture has a pH between 6.0 and 8.0.

6. Method according to claim 1 wherein the detection of growth in step (c) is carried out with a color indicator

7. Method according to claim 6 wherein said color indicator is a pH and/or redox indicator.

8. Method according to claim 1 wherein the test mixture comprises maltose, sucrose or trehalose.

9. Composition comprising Escherichia coli and a color indicator characterized in that the amount of water present in said composition is less than 4% of the total weight of said composition.

10. Composition according to claim 9 further comprising a disaccharide.
